(19) United States
(12) Patent Application Publication
(10) Pub. No.: US 2019/0001051 A1
(43) Pub. Date: Jan. 3, 2019

(54) AUTOMATICALLY COMMUNICATING BETWEEN A NON-MRI COMPATIBLE IV PUMP AND A MRI COMPATIBLE IV PUMP

(71) Applicant: KONINKLIJKE PHILIPS N.V., EINDHOVEN (NL)

(72) Inventors: John CRONIN, BONITA SPRINGS, FL (US); Seth Melvin CRONIN, BONITA SPRINGS, FL (US)

(21) Appl. No.: 16/063,718

(22) PCT Filed: Dec. 30, 2016
(86) PCT No.: PCT/EP2016/082920

§ 371 Date: Jun. 19, 2018

Related U.S. Application Data
(60) Provisional application No. 62/274,052, filed on Dec. 31, 2015.

(51) Int. Cl. A61M 5/142; G06F 19/00; A61M 5/168

(52) U.S. Cl. CPC A61M 5/142; G06F 19/00; A61M 2005/14208; A61M 2205/502; A61M 5/168

(57) ABSTRACT

Methods and systems for automatically communicating information (e.g., patient information, flow rate, etc. . . . ) between a non-MRI compatible IV pump and a MRI compatible IV pump are described herein. Such methods and systems prevent human error from affecting the reprogramming of the IV pumps. Furthermore, automatically clamping infusion lines when a new line is added and locked in also reduces possible human error by removing the need for user input during this process.

### CROSS-REFERENCE TO RELATED APPLICATIONS

The present application claims the priority benefit of U.S. provisional application No. 62/274,052 filed Dec. 31, 2015 and entitled “Automatically Communicating Between a Non-MRI Compatible IV Pump and a MRI Compatible IV Pump,” the disclosure of which is incorporated herein by reference in its entirety.

### BACKGROUND

### Field of Invention

The present invention generally relates to the field of magnetic resonance imaging (MRIs). In particular, the present invention is directed towards facilitating communication between a non-MRI compatible intravenous (IV) pump and a MRI compatible IV pump.

### Description of the Related Art

Magnetic resonance imaging (MRI) is a medical imaging technique that uses radiology to image the autonomy and physiological processes of a patient's body undergoing the technique. Such medical imaging techniques allow medical professionals to characterize the patient's health. MRI scanners can use strong magnetic fields, radio waves, and field gradients to form images of the patient's body.

When a subject undergoes MRI treatment, any intravenous (IV) pumps used to provide IV fluids to the subject must be MRI compatible. However, MRI compatible IV pumps are less common then MRI incompatible IV pumps. Typically, patients are transferred from an MRI incompatible pump to a MRI compatible pump prior to performing a MRI exam. The process of transferring between MRI incompatible pump to a MRI compatible pump includes not only migrating the fluid connection from a first IV pump (associated with the MRI incompatible pump) to a second IV pump (associated with the MRI compatible pump), but also a transfer of various other information such as IV parameters relating to the patient, the drug being administered, and the IV flow rate.

Typically, these parameters being transferred from the MRI incompatible pump to the MRI compatible pump are performed by a user such as a doctor or nurse. Furthermore, these parameters must then be transferred back from the MRI compatible pump to the MRI incompatible pump when the MRI procedure is complete. The existence of these transfers carried out by users allows for user error. Exemplary errors that can occur can be if at either transfer time the parameters were not entered exactly as they were. Furthermore, errors may also occur if the two pumps have different entry methods for inputting the information by the user.

### SUMMARY OF THE CLAIMED INVENTION

A system for facilitating patient and intravenous (IV) pump data to be transferred from a first IV pump to a second IV pump without user intervention is presently claimed. The system includes a processor that executes instructions to perform a search for the first IV pump in order to locate the second IV pump intended for the transfer. Once the second IV pump has been located, the processor executes instructions to transmit the patient and IV pump data associated with the first IV pump to the second IV pump. Such transfer can be performed wirelessly or via a hardwired connection. Furthermore, the system is also capable of automatically clamping infusion lines when lines are added and locked in.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 illustrates a system for communicating between non-MRI compatible IV pumps and MRI compatible IV pumps.

FIG. 2 illustrates another embodiment of a system for communicating MRI compatible IV pumps and MRI compatible IV pumps.

FIG. 3 illustrates a method for implementing the automatic communication between non-MRI compatible IV pumps and MRI compatible IV pumps.

FIG. 4 illustrates another method for implementing the automatic communication between non-MRI compatible IV pumps and MRI compatible IV pumps.

FIG. 5 illustrates a method for implementing the automatic transfer of data from an IV pump to a wireless network.

FIG. 6 illustrates a method for implementing the automatic transfer of data from a wireless network to an IV pump.

FIG. 7 illustrates a method for implementing the automatic integration and storage of data transferred from one IV pump to another IV pump across a network.

FIGS. 8-11 illustrates example graphical user interfaces that may be generated and displayed by the system for communicating MRI compatible IV pumps and MRI compatible IV pumps.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Methods and systems are described herein. Such methods and systems prevent human error from affecting the reprogramming of the IV pumps. Such human error can come into effect, for example, when information is reprogrammed differently from one IV pump to another. Furthermore, automatically clamping infusion lines when a new line is added and locked in also reduces possible human error by removing the need for user input during this process.

It should be noted that the systems and methods described herein may be applicable to transfers of information between any two different IV pumps independent of whether they are non-MRI compatible or MRI compatible. Such systems and methods described below would similarly facilitate transfer of the same sorts of information while preventing human error in these situations as well.

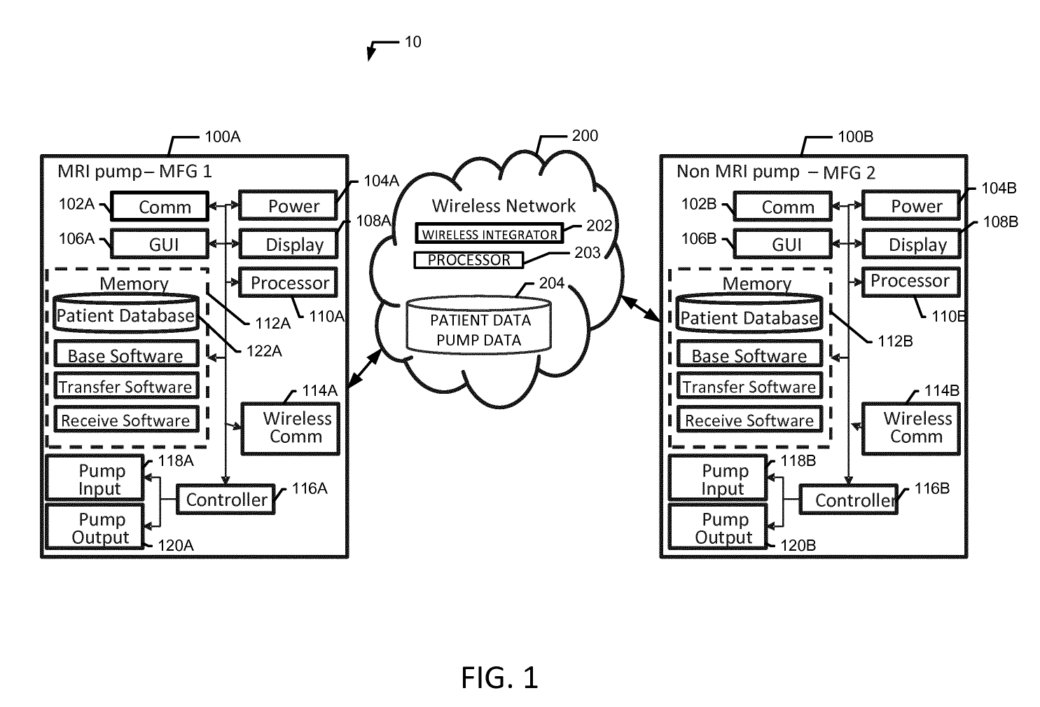

With reference to FIG. 1, a system 10 for communicating between MRI compatible IV pumps 100A and non-MRI compatible IV pumps 100B is illustrated. These two different IV pumps may be connected to each other via a wired or wireless network 200.

For the purposes of the present disclosure, the details for each of the two different IV pumps (e.g., non-MRI compatible and MRI compatible) are illustrated as having similar/same features. However, it should be noted that IV pumps currently available in the market and used, for example, in hospitals, can have more or less features illustrated in the figure. In situations where the two different IV pumps are different in what features they possess, transfer of information between the two IV pumps by a user (e.g., doctor, nurse) can introduce human error.

As illustrated in FIG. 1, exemplary IV pumps may include various features that facilitate in the operation of the IV pump. These features include a communication module 102A-B, power supply 104A-B (e.g., battery), graphical user interface (GUI) 106A-B, display 108A-B, processor 110A-B, memory 112A-B, wireless communication module 114A-B, controller 116A-B, pump inputs 118A-B, and pump outputs 120A-B.

The communication module 102A-B and/or wireless communication module 114A-B are included in order to facilitate communication of the IV pump with other elements of the system. Such communications can be carried out, for example, via wireless means. For example, wireless means of communication that can be carried out by the IV pump to the wireless network can include Wi-Fi, 3G, 4G, LTE, and Bluetooth. It should be noted that other means of communication (e.g., wired, wireless) used by the communication module 102A-B and/or wireless communication module 114A-B known in the art are also possible.

The processor 110A-B of the IV pump may be any computer processor known in the art. The processor 110A-B can be used to carry out the various instructions of the IV pump (e.g., stored in memory 112A-B). In some embodiments, the IV pump may include two or more processors.

The power supply 104A-B may be included to provide power for the operation of the IV pump. The power supply 104A-B may be implemented through the use of a capacitor or a battery. The power supply 104A-B may also be capable of being charged or re-charged using an external power source (e.g., battery charger).

The IV pump may also include a GUI 106A-B. The GUI 106A-B would allow users to interact with the IV pump via graphical icons and visual indicators. For example, the GUI 106A-B could be used to dictate what types of data should be transferred and to which IV pump to transfer the data.

The display 108A-B of the IV pump may be provided so that information can be shown to the user to view. In some embodiments, the display 108A-B may also be a touch screen display that may allow the user to interact with the IV pump.

A controller 116A-B may be included in the IV pump to manage the operation of (and connection) between the IV pump and the various infusion lines that may be connected to the IV pump. The controller 116A-B interacts with the pump inputs 118A-B and the pump outputs 118A-B of the IV pump that dictates how the IV pump uses the infusion line.

The memory 112A-B of the IV pump may include various different types of information. As illustrated in FIG. 1, the memory 112A-B may include a patient database, base software, transfer software, and receive software.

The patient database includes information about the patient associated with the IV pump. Such information may include the patient's name, age, conditions, and parameters associated with the IV pump being used by the patient. The base software may be responsible for the management and operation of the IV pump. The transfer/receive software stored in memory 112A-B corresponds to software used by each IV pump to transmit/receive information to/from the network. Since each IV pump may be different from each other, the software may be capable of transforming or interpreting the data to be transmitted and received into a form that is most suitable or understandable.

Returning to FIG. 1, each of the IV pumps (e.g., MRI-compatible and non-MRI compatible) are connected via a wireless network. As indicated above, the IV pumps are connected to the wireless network via the wireless communication module 114A-B. The wireless network 200 includes both hardware and software components, including but not limited to one or more server, a wireless integrator module 202, one or more processors 203, and a database 204 containing patient data and pump data.

The patient data being transmitted from the first IV pump to the second IV pump may be stored within the wireless network 200. The patient data may be provided to the wireless network 200 via the IV pump's wireless communication module 114A-B. It may also be possible that the patient data may also have been previously stored into the network, for example, by a nurse or doctor. In this way, patient data inputted by the medical professional and being transmitted by the IV pump can be compared to determine if any discrepancies exist. In any case, with the use of the wireless network 200, the transmission of information between the first IV pump to the second IV pump reduces the opportunity for human error to affect the transfer.

Data about each of the IV pumps available, for example, to a hospital, may also be stored within the wireless network (i.e. pump data). Exemplary pump data may include operational information about each IV pump such as its current flow rate, errors and warnings, what drugs are current associated with it and where a particular IV pump is located within the hospital (e.g., currently in use by a particular patient, standby).

It may be possible that the pump data, stored within the network, can be used to locate available and/or compatible IV pumps. In situations where MRI compatible IV pumps are not as numerous, a user (e.g., doctor, nurse) may need to locate where existing MRI compatible IV pumps are and determine which one can be used.

The wireless integrator module 202 facilitates the matching between two different IV pumps so that the patient data and particular pump data associated with the first IV pump can be transferred to a designated second IV pump. In one aspect, the wireless integrator module 202 resides in a wireless network, such as the network 200. The wireless integrator module 202 handshakes with all IV pumps connected to the network, and allows each IV pump to find available IV pumps for transfer of data. The wireless integrator module 202 facilitates the transfer and stores data about the transfer in the secure database 204.

It should be noted that the IV pumps are also capable of being connected to each other via a wired connection. An exemplary wired connection can be implemented via a hardwired connector disposed within transfer tubing used to transfer fluid connections between the two IV pumps. For the purposes of the present disclosure, however, embodiments described herein will reference wireless connections with a wireless network.

Further details regarding how the system carries out the automatic communications between non-MRI compatible IV pumps and MRI compatible IV pumps are provided below.

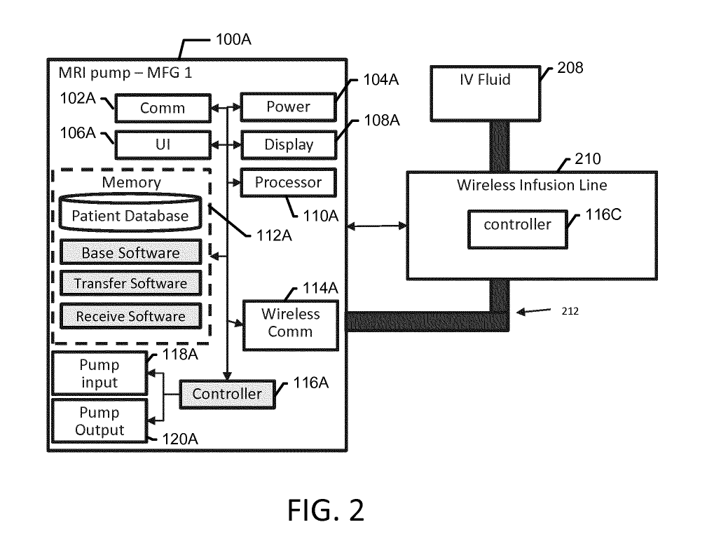

FIG. 2 illustrates an alternative system 100 for communicating between MRI compatible IV pumps and MRI compatible IV pumps. In particular, as shown, IV fluid 208 is provided to the MRI-compatible pump 100A via a wireless infusion line 210 having a controller 116C. The wireless infusion line 210 is in both wireless communication and fluid communication with the MRI pump 100A. The wireless infusion line 210 and associated tubing is used to transfer the IV fluid between two or more pumps. The wireless infusion line 210 also includes an electronic or digital valve that is operatively engaged to the controller 116C. When a command is given, the controller 116C of wireless infusion line 210 automatically closes or opens the valve in the fluid line 212.

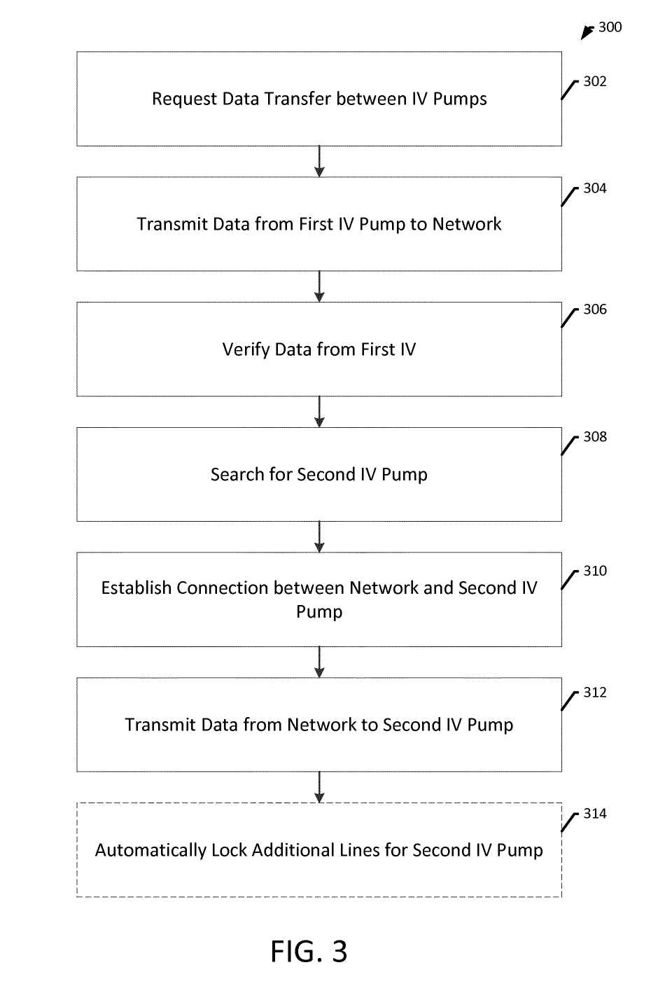

With respect to FIGS. 3-10, a method 300 for implementing the automatic communication between MRI compatible IV pumps 100A and non-MRI compatible IV pumps 100B is shown. As noted above, this method 300 may not be limited to communications just between MRI compatible IV pumps and non-MRI compatible IV pumps. It may be possible, within the spirit of the present disclosure, to expand the method 300 to implement communication between any two IV pumps independent of whether the IV pumps are non-MRI compatible or MRI compatible.

According to one embodiment of the method 300, as shown in FIG. 3, the user (e.g., doctor, nurse) would typically initiate a request, at 302, for data transfer from a first IV pump 100B (e.g., non-MRI compatible pump) to a second IV pump 100A (e.g., MRI compatible pump) via a GUI 106B associated with the first IV pump. The request may include identifying what types of data needs to be transferred (e.g., patient data, pump data), the reason for the transfer, an identity of the second IV pump 100A to be transferred to and if any additional lines need to be added to the second IV pump to complete the transfer.

Once the request for data transfer from the first IV pump has been completed, the first IV pump connects to the network 200 (e.g., wireless network) via the wireless communication module 114A-B. Once connected, the identified data can be transmitted to the network and temporarily stored at 304.

In some cases, the transmitted data may have already been previously inputted by a user (e.g., doctor, nurse). In this case, the wireless network 200 can evaluate/verify, at 306, if there is any sort of discrepancy between the information being provided by the first IV pump and the information previously stored in the network. Any discrepancies may then be notified to the appropriate users (e.g., nurse, doctor).

Once the data from the first IV pump has been transmitted and/or verified, the wireless network 200 can then attempt to search for the identified second IV pump at 308. Presumably, the identified second IV pump is also connected to the wireless network 200. The wireless network 200, via the wireless integrator module 202, can continually poll for the identified second IV pump until it has located the second IV pump. In some embodiments, after a pre-defined period of time, if the identified second IV pump is not found, the wireless network 200 may return information that the second IV pump is not available or is not currently connected to the network for the user (e.g., doctor, nurse) to view on the GUI 106A of the first IV pump.

Once the wireless network 200 finds the identified second IV pump, the connection between the wireless network 200 and the second IV pump is not completed until the user (e.g., doctor, nurse) confirms such connection via the GUI 106B on the second IV pump at 310. In this way, the user can confirm that the identified second IV pump that the wireless network 200 is currently connected to is the correct one. This is to avoid situations where the user (e.g., doctor, nurse) erroneously connects to a different IV pump than to the one intended.

Via the GUI 106B on the second IV pump, the user (e.g., doctor, nurse) confirms that the second IV pump is the correctly identified IV pump (e.g., MRI compatible) for which the data should be transferred to. Once the confirmation is received by the wireless network 200 at 312, the data from the first IV pump is then provided to the identified second IV pump. Such transfer of information does not involve user input thereby reducing the opportunity for human error in transferring the information between the first IV pump to the second IV pump.

Once the data transfer from the first IV pump to the second IV pump is complete, in some embodiments, an initiation sequence at the second IV pump may be requested. This initiation sequence at the second IV pump requests confirmation from the user (e.g., doctor, nurse) that the appropriate IV fluids (e.g., medicine/drugs, solution) are connected to the second IV pump. Once verified, the second IV pump can then begin operation. Furthermore, the initiation sequence signals to the first IV pump that it can terminate operation.

During the confirmation of the appropriate IV fluids to the second IV pump, the user (e.g., doctor, nurse) may be instructed to provide new or additional infusion lines to the second IV pump in order to provide the required IV fluids. Once the infusion lines are provided, the second IV pump (via the wireless network 200) is capable of automatically clamping and incorporating the infusion line to provide the IV fluids to the patient without further user input at 314.

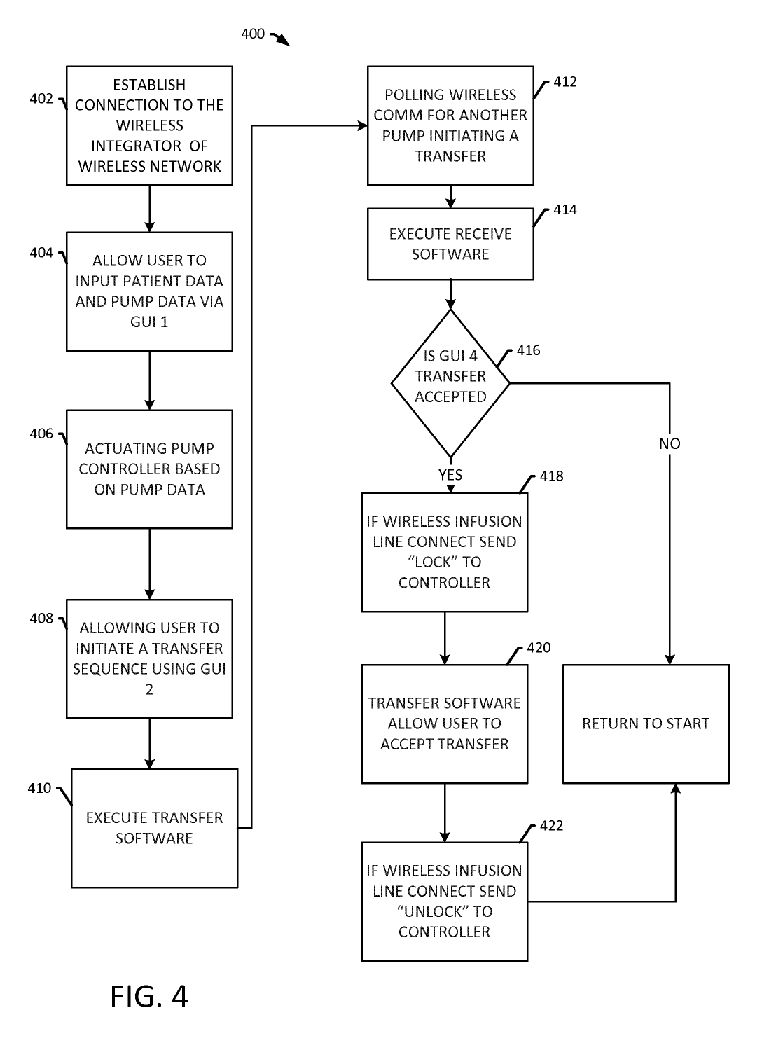

FIGS. 4-11 depict various aspects and features of another embodiment of method 400 to implement the automatic communication between MRI compatible IV pumps 100A and non-MRI compatible IV pumps 100B. In one aspect, the method 400 is performed or caused to be performed by the execution of one or more software applications, such as a base software, a transfer software, and a receive software on one or more processors. While described herein as executing on the processor 110B of a non-MRI compatible pump 100B, a processor of the wireless integrator module 202, and the processor 110A of a non-MRI compatible pump 100A, the software applications or portions thereof may be executed on other computing devices and/or any IV pump to facilitate automatic communication between any combination of MRI compatible pumps, non-MRI compatible pumps, or both.

In various aspects, the method 400 includes a common protocol that could be shared among many manufacturers, allowing a variety of IV pumps, including but not limited to the pumps 100A and 100B as shown in FIGS. 1 and 2, to seamless exchange data over a wireless network 200. In one aspect, as shown in FIG. 2, infusion line controllers associated with the various IV pumps will lock and unlock IV pump during transfer to ensure safety.

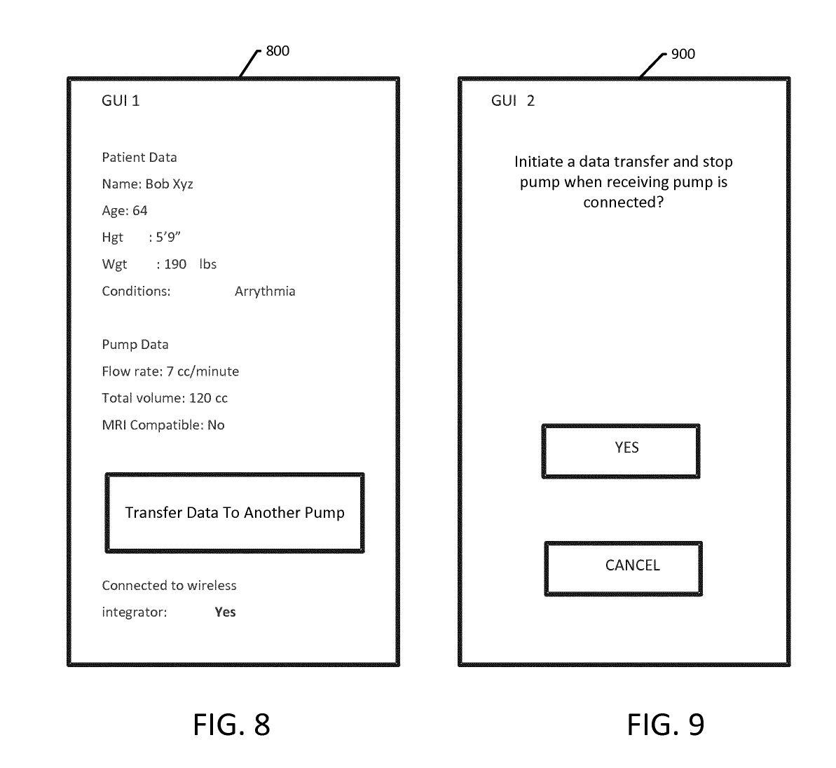

According to one aspect of the method 400, the base software establishes a connection to the wireless integrator module 202 of wireless network 200 at step 402. At step 404, a user (e.g., doctor, nurse, or other practitioner) inputs patient data and pump data via a patient GUI, such as the non-limiting example GUI 1, generally indicated as 800, in FIG. 8. The patient GUI 800 is displayed on the display device 106B of the pump 100B.

At step 406, the pump controller 116B is actuated based on pump data, while at step 408, the user is allowed to initiate a transfer sequence using a transfer GUI. A non-limiting example of the transfer GUI is provided as GUI 2 generally indicated as 900, in FIG. 9. The transfer GUI 900 is displayed on the display device 106B of the pump 100B. In one aspect, the transfer GUI 900 may require the user to stop the pump 100B.

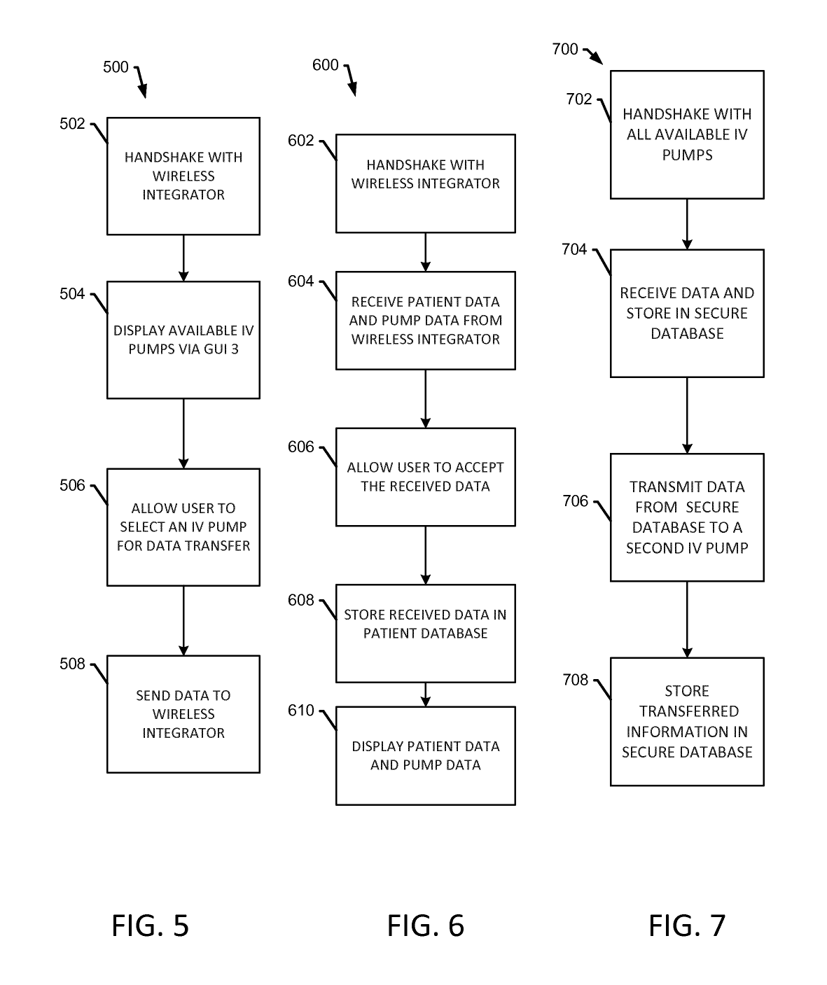

At step 410, a transfer software module is executed. The process performed or the process caused to be performed by the execution of the transfer software module at the processor 110B, is shown and described with references to FIG. 5. As shown the transfer software module carries out a process 500 for automatically transferring data between one or more pumps.

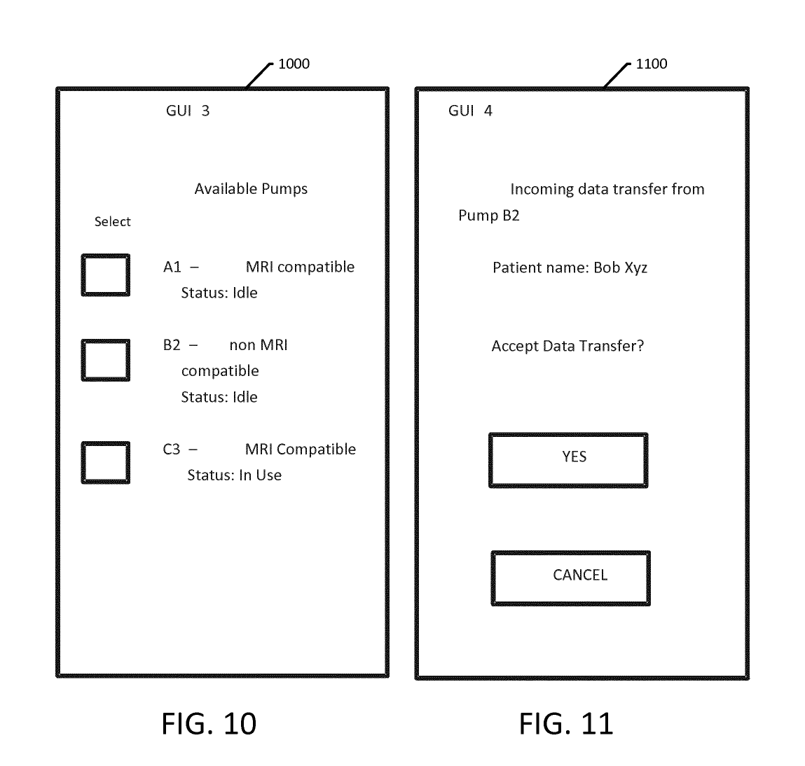

For example, at step 502, the IV pump 100B performs a handshake with the wireless integrator module 202, which provides the transfer software module with data about the available IV pumps. At step 504, the processor 110B generates and displays an availability GUI to identify the available IV pumps. A non-limiting example of the availability GUI is provided as GUI 3 generally indicated as 1000, in FIG. 10. The availability GUI 900 is displayed on the display device 106B of the pump 100B.

At step 506, the user is allowed to select an IV pump for data transfer at the availability GUI 900. The IV pump selection with along with patient data and pump data is transmitted to the wireless integrator module 202, at step 508.

Referring again to FIG. 4, after receiving an IV pump selection and the accompanying data, the wireless integrator module 202 polls the wireless communication module of the selected pump (e.g. wireless communication module 114A), at step 412. At step 414, a receive software module is executed at a receiving IV pump, such as the pump 100A. The process performed or the process caused to be performed by the execution of the receive software module at the processor 110A, is shown and described with references to FIG. 6. As shown the receive software module carries out a process 600 for automatically receiving data from one or more pumps.

For example, at step 602 the IV pump 100A performs a handshake with the wireless integrator module 202. At step 604, the pump 100A receives patient data and pump data from wireless integrator module 202. At step 606, the processor 110A generates and displays an acceptance GUI to confirm acceptance of the data transfer. A non-limiting example of the acceptance GUI is provided as GUI 4 generally indicated as 1100, in FIG. 11. The acceptance GUI 1100 is displayed on the display device 106A of the pump 100A. In one embodiment, the acceptance GUI 1100 includes an agreement indication input to indicate that the data is correct to download. In another embodiment, the GUI 1100 may indicate to the user to load the desired IV fluid, thus the second IV pump 100A may resume pumping in the same manner as that pumping that was halted at the first pump 100B.

At step 608, the IV pump 100A stores the received data in a local patient database 122A. At step 610, the processor 110A generates and displays a patient GUI, similar to GUI 1, generally indicated as 800, in FIG. 8. The patient GUI 800 is displayed on the display device 106A of the pump 100A.

Returning again to FIG. 4, the base software determines if the data transfer described with reference to step 606 and FIG. 6, is accepted. If the data transfer is accepted, then a lock command is generated at step 418 and transmitted to the controller 116C of the wireless infusion line 210 in communication with the pump 100A. This halts the flow of IV fluid 208 to ensure patient safety.

At step 420, an instance of the transfer software module executes at the pump 100B to confirm acceptance of the data transfer. Once the transfer is also accepted or confirmed at the sending pump 100B, an unlock command is generated at step 422 and transmitted to the controller 116C of the wireless infusion line 210. Conversely, if the data transfer is not accepted at step 416, the method 400 is halted and a new connection is established, or alternatively, the connection made at step 402 is reestablished.

FIG. 7 depicts a flowchart of an integrator method 700 performed or caused to be performed by the wireless integrator module 202 executing on the processor 203. The various steps of the method 700 are performed as necessary when performing the method 400 and related sub-methods or sub-processes described with reference to FIGS. 3-6. As such, the actions described as steps 702-708 may be performed in any order.

In one aspect, the wireless integrator module 202 performs handshakes with all available IV pumps at step 702. In various embodiments, communication between the wireless integrator module 202 and each of the IV pumps 100A-B preferably occurs using Bluetooth hardware configured for infrared (IR) transmission or near field communication (NFC), or a combination thereof. In this particular embodiment, the shorter range of IR and NFC, as compared to general radio frequency or wireless local area network (WLAN) communication is preferred to prevent unintended pairings between the integrator and the IV pumps devices. Thus, according to one aspect, the ability and availability of the pumps 100A-B to communicate with the wireless integrator module 202 is determined at least in part by the distance between the pumps and the integrator module.

At step 704, the wireless integrator receives the IV pump selection, patient data, and pump data from the first IV pump 100B and stores the data in the secure database 204. At step 706, the integrator 202 transmits patient data and pump data from the secure database 204 to the second IV pump 100A based on the user selection and the stores the transferred information in the secure database 204 at step 708.

The various computing devices disclosed herein include computer readable media (CRM) in each respective memory on which the described applications and software are stored. The computer readable media may include volatile media, nonvolatile media, removable media, non-removable media, and/or another available medium that can be accessed by the respective processors. By way of example and not limitation, the computer readable media comprises computer storage media and communication media. Computer storage media includes non-transitory storage memory, volatile media, nonvolatile media, removable media, and/or non-removable media implemented in a method or technology for storage of information, such as computer/machine-readable/executable instructions, data structures, program modules, or other data. Communication media may embody computer/machine-readable/executable instructions, data structures, program modules, or other data and include an information delivery media or system, both of which are hardware.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

1-4. (canceled)

5. A system for automatically communicating information between IV pumps, the system comprising:
a first IV pump comprising a first memory and a first processor, the first processor programmed to:
receive a communication identifying available IV pumps;
transmit a request to data transfer from the first IV pump to a second IV pump selected from the communication identifying available IV pumps to a third processor;
receive selection input identifying the second IV pump;
transmit patient data and pump data to the third processor; and
receive a communication to terminate operation of the first IV pump;
the second IV pump comprising a second processor and a second memory, the second processor to:
receive the patient data and the pump data from the third processor;
receive acceptance input indicating acceptance of the patient data and pump data received from the third processor; and
storing the received patient data and pump data in the second memory; and
a network server comprising a third memory and the third processor, the third processor to:
establish communication with the first IV pump and the second IV pump;
receive the patient data and the pump data from the first processor;
store the patient data and the pump data in the third memory; and
transmit the patient data and the pump data to the first processor.

6. The system of claim 5, wherein the first IV pump further comprises a first display device.

7. The system of claim 6, further comprising the first processor to display a graphical user interface comprising the patient data at the first display.

8. The system of claim 6, further comprising the first processor to display a graphical user interface for initiating the transmission of the patient data and pump data.

9. The system of claim 6, further comprising:
the first processor to:
receive a communication identifying IV pumps available to receive the patient data and the pump data display; and
generate a graphical user interface for display at the first display device, the graphical user interface comprising a listing of the IV pumps available to receive the patient data and the pump data display.

10. The system of claim 5, wherein the second IV further comprises a second display device.

11. The system of claim 10, further comprising the second processor to display a graphical user interface comprising the patient data and the pump data received from the third processor at the second display.

12. The system of claim 5, wherein each of the first IV pump, the second IV pump, and network server comprises at least one of a respective Bluetooth communication device or a respective near-field communication device.

13. The system of claim 12, wherein communication between the first IV pump and the network server, communication between the second IV pump and the network server, or both is determined at least in part by a first distance between the first IV pump and the network server and a second distance between the second IV pump and the network.

14. A method for automatically communicating information between a plurality of IV pumps, the method comprising:
receive a communication identifying a plurality of available IV pumps;
establishing, by a first processor, communication with a first IV pump of the plurality of IV pumps;
receiving, by a second processor, patient data and pump data;
generating, by the first processor, a request to actuate a controller of the first IV pump based on the received pump data;
receiving, by the first processor, a request to initiate transfer of the patient data and pump data from the first IV pump to at least one second pump of the plurality of IV pumps;
receiving, by the first processor, the patient data and pump data from the second processor;
polling, by the first processor, to establish communication with a third processor, wherein the at least one second pump includes the third processor;
storing, by the first processor, the patient data and pump data in a database;
transmitting, by the first processor, the patient data and pump data to the third processor;
determining, by the first processor, that the patient data and pump data is accepted at the third processor;
receiving, by the first processor, an acceptance request from the third processor;
generating, by the first processor, a command to terminate a pumping operation to the second processor;
receiving, by the first processor, confirmation of the termination of the pumping operation;
generating, by the first processor, a command to initiate another pumping operation at the third processor; and
polling, by the first processor, to re-establish communication with the third processor when the patient data and pump data is not accepted at the third processor.

15. The method of claim 14, further comprising:
transmitting, by the first processor, a list of available IV pumps of the plurality of IV pumps to the second processor;
receiving, by the second processor, a selection of a second IV pump of the plurality of IV pumps, wherein the second IV pump was identified in the list of available IV pumps;
receiving, by the second processor, the command to terminate the pumping operation; and
generating, by the second processor, the confirmation of the termination of the pumping operation.

16. The method of claim 14, further comprising:
receiving, by the third processor, the patient data and pump data from the first processor;
generating, by the third processor, the acceptance request;
storing, by the third processor, the patient data and pump data in a local database; and
displaying, by the third processor, the patient data and pump data at a display device.

17. A non-transitory computer-readable storage medium, having embodied thereon a program executable by a processor to perform the method of claim 10.
